(19) United States
(12) Patent Application Publication
(10) Pub. No.: US 2017/0191037 A1
(43) Pub. Date: Jul. 6, 2017

(54) METHOD AND CONTAINER FOR CULTURING UNDIFFERENTIATED INDUCED PLURIPOTENT STEM CELL

(71) Applicant: KOREA INSTITUTE OF SCIENCE AND TECHNOLOGY, Seoul (KR)

(72) Inventors: So Yeon KIM, Seoul (KR); Eun Gyeong YANG, Seoul (KR); Myung-Han YOON, Gwangju (KR); Hyunju KIM, Seoul (KR)

(21) Appl. No.: 15/224,499

(22) Filed: Jul. 29, 2016

(30) Foreign Application Priority Data
Dec. 30, 2015 (KR) 10-2015-0189830

(51) Int. Cl. C12N 5/074; C12N 5/00

(52) U.S. Cl. CPC C12N 5/0696; C12N 5/0075; C12N 2535/00; C12N 2531/00

(57) ABSTRACT

Provided are a method of culturing an induced pluripotent stem cell and a method of forming a spheroid of the induced pluripotent stem cell. According to an embodiment of the present inventive concept, the induced pluripotent stem cell may have improved stemness.

### CROSS-REFERENCE TO RELATED APPLICATION

This application claims the benefit of Korean Patent Application No. 10-2015-0189830, filed on Dec. 30, 2015, in the Korean Intellectual Property Office, the disclosure of which is incorporated herein in its entirety by reference.

### BACKGROUND

1. Field

One or more embodiments relate to a container for culturing induced pluripotent stem cells, a method of culturing induced pluripotent stem cells in an undifferentiated state, and a method of forming a spheroid of induced pluripotent stem cells (iPSCs).

2. Description of the Related Art

Recently, the utilization of stem cells have been cautiously proposed for biomedical applications, particularly in regenerative medicine with a cell based treatment. To utilize stem cells in the clinical application, it is required to find a safe and cost-effective source of stem cell. In this regard, iPSCs are getting the spotlight as a source of stem cell therapy. iPSCs can be obtained by reprogramming differentiated cells using dedifferentiation-inducing factors. Thus, without somatic cell nuclear transfer, patient immune-compatible pluripotent derived cell lines, thereby avoiding the immune rejection when used in clinical tirals, can be produced. Since neither eggs nor embryos are used to produce iPSCs, iPCS are advantageous to having no bioethical controversy or religious issues. However, a method of producing iPSCs through an existing reprogramming process has a disadvantage of very low production efficiency, and due to use of animal-derived feeder cells in the process of dedifferentiation induction and proliferation, there is a limitation to applying iPSCs to actual clinical trials in terms of safety. Therefore, there is a need for a method of culturing iPSCs in a way of having high viability while maintaining differentiation potency in a dedifferentiated state without using feeder cells.

Traditionally, three-dimensional (3D) embryonic bodies (EBs) or spherical colonies have been generated by growing stem cells in hanging drops or supporting suspension cultures in spinner flasks to impede cellular adhesion to a substrate surface. However, the issues on viability, uniformity, and functionality associated with such conventional stem cell cultures have spurred many researchers to search for novel culture protocols for producing well-defined spherical colonies. Notably, the utilization of unconventional substrates with controlled biophysical cues including substrate modulus and surface topography has received growing interest, as exemplified by microwells and micropatterns as alternative substrates for functional EB formation.

In this regard, the present inventors put effort into developing a method of culturing iPSCs with high proliferation efficiency, and consequently, found that the viability and stemness of iPSCs can be both improved in the case a nanostructure is utilized in culturing the iPSCs, thereby achieving the present inventive concept.

### SUMMARY

One or more embodiments include a container for culturing induced pluripotent stem cells (iPSCs).

One or more embodiments include a method of culturing iPSCs so as to have high proliferation efficiency in an undifferentiated state and have excellent stemness.

One or more embodiments include a method of forming a spheroid of iPSCs.

### BRIEF DESCRIPTION OF THE DRAWINGS

These and/or other aspects will become apparent and more readily appreciated from the following description of the embodiments, taken in conjunction with the accompanying drawings in which:

FIG. 1 is a scanning electron microscopic (SEM) images of silicon (Si) wafer and vertical silicon nanocolumn arrays (vSNAs) with three different lengths (e.g., short, medium, and long) (based on a scale bar of 1 μm);

FIGS. 2A-2B show spherical colonies of induced pluripotent stem cells (iPSCs) that are produced by vSNAs having three different lengths (short, medium, and long) after three days of culturing, and more particularly, FIG. 2A shows carboxyfluorescein diacetate (CFDA)-staining images of spherical colonies of iPSCs that are produced on an attachment factor (AF)-coated plate, a silicon (Si) wafer, and vSNAs, for three days (based on a scale bar of 200 μm), and FIG. 2B is a graph (* P<0.05) showing quantitative results of the number of spherical colonies (having a diameter of about 200 μm or more) of iPSCs that are cultured on various surfaces;

FIG. 3A shows SEM images showing a Si wafer and four vSNAs each having a different density (i.e., vSNA1, vSNA2, vSNA3, and vSNA4) (based on a scale bar of 1 μm); FIG. 3B shows images showing shapes of spherical colonies of iPSCs on the third day of culturing on the AF-coated plate, the Si wafer and the four vSNAs, wherein the images are obtained by staining the spherical colonies with CFDA for fluorescence detection (based on a scale bar of 200 μm); and FIG. 3C is a graph (* P<0.05) showing quantitative results of the number of spherical colonies (having a diameter of 200 μm or more) of iPSCs that are cultured on various substrates;

FIG. 4 shows three-dimensional reconstructions of confocal microscope images of iPSCs that are each cultured on an AF-coated plate, a Si-wafer, and a vSNA (e.g., vSNA4);

FIGS. 5A-5C show morphological characteristics of iPSC spherical colonies that are produced on different surfaces, and more particularly, FIG. 5A shows confocal microscopic images of stained cells on the fifth day of culturing, wherein living cells in spheroids are stained with CFDA (green) while dead cells are stained with PI (red) (based on a scale bar of 100 μm); FIG. 5B shows diameter distribution of spherical colonies produced on the fifth day of culturing on a petri dish and a vSNA (e.g., a vSNA4) (based on a scale bar of 200 μm. P<0.005); and FIG. 5C shows immunoblotting results of E-cadherin and N-cadherin expression of iPSC spheroids produced on the fifth day of culturing on the petri dish and vSNAs;

FIGS. 6A-B show adhesion properties of iPSCs that are cultured on various surfaces, and more particularly, FIG. 6A shows immuno-fluorescent images of iPSCs that cultured on various surfaces for 24 hours, wherein focal adhesion vinculin (green) and cytoskeletal actin filament (red) of the cultured iPSCs are visualized simultaneously under a confocal microscope, and the nucleus of the cultured iPSCs is stained with Hoechst 33342 (blue) (based on a scale bar of 10 μm); and FIG. 6B is a graph showing quantitative results of cell attachment, wherein iPSCs are cultured on various surfaces for 24 hours, and a fluorescent image of cells that are attached to a vSNA (e.g., a vSNA4) is designated as 1 (based on a scale bar of 200 μm. P<0.005);

FIGS. 7A-7C show expression levels of pluripotent markers within iPSCs that are cultured on various surfaces for 5 days, and more particularly, FIG. 7A is a graph showing AP activity of iPSCs (*, P<0.05; P<0.005); FIG. 7B is a graph showing results of semi-quantitative RT-PCR performed on pluripotent stem cell markers (e.g., Nanog, Oct-3/4, and Sox-2); and FIG. 7C is a graph showing immunoblotting results of pluripotent stem cell markers (e.g., Nanog, Oct-3/4, and Sox-2);

FIG. 8A shows FACS analysis results of SSEA-1 expression in iPSCs; and FIG. 8B shows results of semi-quantitative RT-PCR performed on naive stem cell markers (e.g., Rex-1 and Stella);

FIGS. 9A-9B show differentiation capability of iPSCs that are cultured on a vSNA (e.g., a vSNA4), and more particularly, FIG. 9A shows results of semi-quantitative RT-PCR performed on markers for three germ layers in iPSCs that are differentiated in EB medium for 14 days after being cultured on a petri dish and a vSNA (e.g., a vSNA4) for 5 days, wherein Nestin and TP63/TP73L are markers for an ectoderm, AFP, GATA-4, PDX-1/IPF1, and HNF-3b/FoxA2 are markers for an endoderm, and Brachyury is a marker for a mesoderm; and FIG. 9B shows results of semi-quantitative RT-PCR performed on pluripotent stem cell markers (e.g., Nanog and Oct-3/4) in iPSCs that are differentiated on various surfaces for 14 days;

FIG. 10 shows immuno-fluorescent images of three germ layers of iPSCs that are differentiated on a petri dish and a vSNA (e.g., a vSNA4) for 14 days, wherein an ectoderm (white; Tuj1), an endoderm (green: AFP), and a mesoderm (red: SMA) are each visualized by using a confocal microscope and the nuclei thereof are each stained with Hoechst 33342 (blue) (based on a scale bar of 50 μm); and

FIGS. 11A-B are diagrams describing that a vSNA according to the present inventive concept can be recycled as a culture container for culturing iPSCs and can be applicable for massive culture of iPSCs, and more particularly, FIG. 11A shows fluorescent microscopic images and DIC images of iPSC spheroids that are obtained as follows: iPSCs are cultured on a vSNA to form colonies, and a washing process is performed thereon to remove the colonies, and then, new iPCSs are cultured again on the same vSNA to form iPSC spheroids (as shown in FIG. 11A, based on a scale bar of 200 μm.); and FIG. 11B shows images of a vSNA that is manufactured in a large scale representative and stitched fluorescence microscopic images of iPSCs on a large size vSNA substrate after 3 days of culture (based on a scale bar of 5 mm and 1 mm).

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Additional aspects will be set forth in part in the description which follows and, in part, will be apparent from the description, or may be learned by practice of the presented embodiments.

According to one or more embodiments, a culture container for culturing induced pluripotent stem cells includes a nanostructure array.

The culture container according to the present inventive concept may include a nanostructure array.

The term “nanostructure” used herein refers to a nano-scale (10⁻⁹) object. In general, the nanostructure refers to an object including every structure having a size in a range of about 0.1 to about 100 nanometers (nm), and the term can be used interchangeably with the term ‘nanoconstruct’. The nanostructure may include, for example, a nanorod, a nanotube, a nanowall, or a nanowire, and in some embodiments, the nanostructure may be a nanocolumn. The term “nanocolumn” used herein refers to a columnar-shaped structure having a diameter in a unit of nanometers, that is, the diameter of the nanocolumn may be in a range of about less than 1 nanometer to about several hundred nanometers. A top of the nanocolumn may have a flat shape, and due to such a flat shape, cells can reside, without penetrating cell membranes, on the nanocolumn of the nanostructure array according to the present inventive concept. In this regard, the nanocolumn according to the present inventive concept is different from a nanocolumn utilizing the cell membrane penetration properties according to the related art.

The term “nanostructure array” used herein refers to a plurality of nanostructures that are arranged in a certain rule or in a random manner. The nanostructure array may refer to, for example, a plurality of nanostructures that are spaced apart at random intervals on a substrate, and in some embodiments, may refer to a plurality of nanocolumns that are spaced apart at random intervals on a substrate. Here, the number of nanostructures or nanocolumns is 2 or more. That is, the nanostructure array may specifically refer to 2 or more nanocolumns that are spaced apart at random intervals on a substrate. Although the 2 or more nanocolumns may vertically be on a substrate, embodiments are not limited thereto. In some embodiments, the 2 or more nanocolumns may be on a substrate at an angle having no influence on culturing of induced pluripotent stem cells.

A length of each nanocolumn of the nanostructure array according to the present inventive concept may be about 1.5 μm or less, for example, may be in a range of about 0.5 μm to about 1.5 μm, about 0.5 μm to about 1.4 μm, about 0.5 μm to about 1.3 μm, about 0.5 μm to about 1.2 μm, or about 0.5 μm to about 1.0 μm. In some embodiments, the length of each nanocolumn may be in a range of about 0.5 μm to about 1.0 μm. In some embodiments, it is confirmed that a culture container including the nanocolumns each having a length of about 1.5 μm or less involves formation of a large number of spheroids of induced pluripotent stem cells, compared to a culture container including nanocolumns each having a length greater than about 1.5 μm.

A density of the nanocolumns of the nanostructure array according to the present inventive concept may be, for example, about 150/100 μm² or more, about 200/100 μm² or more, 250/100 μm² or more, about 300/100 μm² or more, about 350/100 μm² or more, about 400/100 μm² or more, about 450/100 μm² or more, or about 500/100 μm² or more (where density=the number of nanocolumns/area of nanostructure array). In some embodiments, the density of the nanocolumns may be in a range of about 150/100 μm² to about 1,000/100 μm², about 200/100 μm² to about 1,000/100 μm², about 250/100 μm² to about 1,000/100 μm², about 300/100 μm² to about 1,000/100 μm², about 350/100 μm² to about 1,000/100 μm², about 400/100 μm² to about 1,000/100 μm², about 450/100 μm² to about 1,000/100 μm², or about 500/100 μm² to about 1,000/100 μm². In some embodiments, it is confirmed that, in a culture container including the nanocolumns at a high density (for example, a density of about 150/100 μm² or more), a shape of spheroids of induced pluripotent stem cells is more like a sphere, and a diameter of colonies of induced pluripotent stem cells formed therein increases. In addition, it is confirmed that, a greater number of spheroids having a diameter of about 200 μm or more are formed in a culture container including the nanocolumns at a higher density than spheroids formed in a culture container including the nanocolumns at a low density or in a flat culture container (i.e., a container not including nanocolumns).

In some embodiments, the nanocolumns of the nanostructure array may have a length in a range of about 0.5 μm to about 1.5 μm and a density in a range of about 150/100 μm² to about 1,000/100 μm² (where density=the number of nanocolumns/area of nanostructure array).

A region in contact with cells in the nanocolumn, i.e., a top of the nanocolumn, may have a flat shape. In this regard, the nanocolumn of the nanostructure array does not allow cells to penetrate therethrough, but rather enhance cell-to-cell interaction of induced pluripotent stem cells.

In some embodiments, the diameter of the nanocolumn may be in a range of about 50 nm to about 500 nm, about 60 nm to about 400 nm, about 70 nm to about 300 nm, about 100 nm to about 300 nm, about 100 nm to about 250 nm, or about 100 nm to about 200 nm.

The nanostructure array may be on any surface of the culture container, and in some embodiments, may be on a bottom surface of the culture container.

The nanostructure may be formed of a non-cytotoxic material, and for example, the material may be at least one selected from the group consisting of silicon (Si), ZnO, GaAs, InP, InAs, nickel (Ni), platinum (Pt), gold (Au), SiO₂, and TiO₂. In some embodiments, the material may be Si.

The term “induced pluripotent stem cell (iPSC)” used herein refers to a cell having pluripotency through dedifferentiation of differentiated cell (for example, a somatic cell), and is also designated as a ‘dedifferentiated stem cell’. The iPSCs used in the present inventive concept may be, for example, prepared by using somatic cells derived from mammals including humans, and more particularly, may be prepared by utilizing a dedifferentiation technique on human somatic cells. A dedifferentiation factor may include, for example, Oct4, Sox, Klf, Myc, Lin28, Nanog, or a combination thereof, but is not limited thereto.

The nanostructure array included in the culture container may have a surface modified so as to have a chemical functional group. For example, a surface of the nanostructure array may be coated with a compound so that a positively-charged amine group may be formed on the surface of the nanostructure array. In some embodiments, a surface of the nanostructure array may be coated with 3-aminopropyltrimethoxysilane (APTMS).

In addition, the culture container may undergo an additional treatment, such as a plasma treatment, an ozone treatment, or other coating treatments using chemicals, in terms of improving proliferation and viability of the iPSCs.

The culture container for culturing the iPSCs according to the present inventive concept may be used to culture the iPSCs under an undifferentiated condition in the absence of a feeder layer. The culturing of the iPSCs under an undifferentiated condition refers to culturing of the iPSCs having pluripotency or stemness maintained therewith while the iPSCs are not differentiated yet. In addition, the culture container may be used to improve pluripotency or stemness of the iPSCs.

In some embodiments, the iPSCs cultured in the culture container are found to exhibit improved stemness and show a decrease in cell death in spherical colonies of the iPSCs. The term “stemness” used herein refers to a characteristic of a stem cell, which is distinguishable from a somatic cell, in that the stem cell is capable of being differentiated into two or more cells while having self-replication ability. That is, stemness refers to characteristics of the stem cell including both a capability of self-replication through a cell division and capability of pluripotency for differentiation into various cells. In this regard, the iPSCs cultured in the culture container of the present inventive concept may have improved stemness, thereby being more suitable as a therapeutic agent in cell therapy.

In addition, the culture container for culturing the iPSCs may be used to form a spheroid of the iPSCs.

The expression “spheroid of iPSCs” used herein refers to an assembly of iPSCs, and more particularly, to a spherically-shaped cell group formed by undifferentiated iPSCs that are densely packed. The term “spheroid” used herein can be used interchangeably with the term ‘spherical colony’.

In addition, the culture container for culturing the iPSCs may be used to increase cell-to-cell interactions of the iPSCs. The culture container according to the present inventive concept may enhance cell-to-cell interactions of the iPSCs, thereby enabling formation of large and dense spheroids of the iPSCs.

In addition, the nanostructure array in the culture container for culturing the iPSCs according to the present inventive concept may be recycled. In some embodiments, it is confirmed that, when the nanostructure array that has been previously used for culturing the iPSCs and forming spheroids of the iPSCs is cleaned by a washing process and is used for culturing new iPSCs, the resulting iPSCs involve formation of large and dense spherical colonies of the iPSCs thereof.

In addition, the culture container for culturing the iPSCs according to the present inventive concept may be a large-area cell culture container. In some embodiments, it is confirmed that, on the nanocolumns of the nanostructure array having an up-graded scale according to a culture dish having a diameter 100 mm, the iPSCs are cultured in an undifferentiated state and spheroids of the iPSCs are well formed. In this regard, the culture container according to the present inventive concept may be suitable for culturing a stem cell, such as an iPSC, which is required in a large amount as a therapeutic agent in cell therapy.

According to one or more embodiments, a method of culturing iPSCs in an undifferentiated state includes culturing the iPSCs on a nanostructure array.

Regarding the method of culturing the iPSCs in an undifferentiated state, the nanostructure is the same as described in connection with the culture container above.

In some embodiments, the nanostructure may be a nanocolumn. A length of the nanocolumn may be about 1.5 μm or less, for example, may be in a range of about 0.5 μm to about 1.5 μm, about 0.5 μm to about 1.4 μm, about 0.5 μm to about 1.3 μm, about 0.5 μm to about 1.2 μm, or about 0.5 μm to about 1.0 μm. In some embodiments, the length of the nanocolumn may be in a range of about 0.5 μm to about 1.0 μm. In addition, a density of the nanocolumn in the nanostructure array may be, for example, about 150/100 μm² or more, about 200/100 μm² or more, about 250/100 μm² or more, about 300/100 μm² or more, about 350/100 μm² or more, about 400/100 μm² or more, about 450/100 μm² or more, or about 500/100 μm² or more (where density=the number of nanocolumns/area of nanostructure array). In some embodiments, the density of the nanocolumn may be, for example, in a range of about 150/100 μm² to about 1,000/100 μm², about 200/100 μm² to about 1,000/100 μm², about 250/100 μm² to about 1,000/100 μm², about 300/100 μm² to about 1,000/100 μm², about 350/100 μm² to about 1,000/100 μm², about 400/100 μm² to about 1,000/100 μm², about 450/100 μm² to about 1,000/100 μm², or about 500/100 μm² to about 1,000/100 μm².

The culturing of the iPSCs may be performed in the culture container including the nanostructure array. The culturing of the iPSCs may be performed according to a known culturing method of iPSCs in the art, or according to a general culturing method of iPSCs that can be easily derived and modified by one of ordinary skill in the art. For example, a medium for culturing iPSCs is added to the culture container including the nanostructure array according to the present inventive concept, and then, iPSCs are seeded onto the medium to allow proliferation of the iPSCs for a certain time period. The medium used herein may be a known medium used for the iPSCs in the art. In addition, the culturing of the iPSCs may be performed by using a technique that is known in the art or that can be easily derived for culturing iPSCs, the technique including steps associated with culturing procedures, adjustment of medium compositions, and addition of medium additives.

The method of culturing the iPSCs in an undifferentiated state according to the present inventive concept may be performed by culturing the iPSCs in the absence of a feeder layer while maintaining an undifferentiated state of the iPSCs. The term “feeder layer” used herein refers to a cell layer cultured together for the purpose of inducing proliferation of stem cells. A culturing method using such a feeder layer is generally used in the art to culture stem cells in an undifferentiated state. In general, the feeder layer is derived from mouse fibroblasts. However, due to problems with xenoinfection, the culturing method using mouse fibroblasts may involve a risk in clinical use associated with human iPSCs. Therefore, the method of culturing the iPSCs according to the present inventive concept may solve disadvantages of xenoinfection that may appear in a known culturing method of stem cells in an undifferentiated state in the related art.

The resulting iPSCs cultured by using the culturing method according to the present inventive concept may exhibit improved stemness. In addition, the resulting iPSCs may show a decrease in cell death and exhibit increased viability in the spherical colonies thereof.

In some embodiments, the iPSCs cultured in the absence of supporting cells on the nanocolumns of the nanostructure array by using the culturing method according to the present inventive concept are subjected to identification of a shape of the cultured iPSCs and expression of pluripotency markers. Consequently, by comparing the iPSCs cultured on the nanocolumns of the nanostructure array with iPSCs cultured on a petri dish, which is a culture container typically used in the art, and a Si-wafer, which does not include any structure, it is confirmed that the iPSCs cultured on the nanocolumns of the nanostructure array by using the culturing method according to the present inventive concept densely form spherical colonies thereof, and furthermore, maintain the expression of Nanog gene at a high level. Furthermore, it is confirmed that the iPSCs cultured on the nanocolumns of the nanostructure array by using the culturing method according to the present inventive concept have improved viability.

In addition, it is confirmed that, when the iPSCs cultured by using the culturing method according to the present inventive concept are differentiated, the iPSCs are well differentiated into 3 germ layers. Therefore, the culturing method according to the present inventive concept involves culturing of iPSCs in an undifferentiated state while maintaining not only pluripotency, but also differentiation potency.

According to one or more embodiments, a method of forming spheroids of iPSCs includes culturing the iPSCs in a nanostructure array.

Regarding the method of forming the spheroids of the iPSCs, the nanostructure array is the same as described in connection with the culture container above.

In some embodiments, the nanostructure may be a nanocolumn. A length of the nanocolumn may be about 1.5 μm or less, for example, may be in a range of about 0.5 μm to about 1.5 μm, about 0.5 μm to about 1.4 μm, about 0.5 μm to about 1.3 μm, about 0.5 μm to about 1.2 μm, or about 0.5 μm to about 1.0 μm. In some embodiments, the length of the nanocolumn may be in a range of about 0.5 μm to about 1.0 μm. In addition, a density of the nanocolumn in the nanostructure array may be, for example, about 150/100 μm² or more, about 200/100 μm² or more, about 250/100 μm² or more, about 300/100 μm² or more, about 350/100 μm² or more, about 400/100 μm² or more, about 450/100 μm² or more, or about 500/100 μm² or more (wherein density=the number of nanocolumn/area of nanostructure array). In some embodiments, the density of the nanocolumn in the nanostructure array may be, for example, in a range of about 150/100 pmt to about 1,000/100 μm², about 200/100 μm² to about 1,000/100 μm², about 250/100 μm² to about 1,000/100 μm², about 300/100 μm² to about 1,000/100 μm², about 350/100 μm² to about 1,000/100 μm², about 400/100 μm² to about 1,000/100 μm², about 450/100 μm² to about 1,000/100 μm², or about 500/100 μm² to about 1,000/100 μm².

When the iPSCs are cultured in the nanostructure array by using the method of forming the spheroids of the iPSCs according to the present inventive concept, there may be enhanced cell-to-cell interaction of the iPSCs, thereby affecting the formation of the spheroids of the iPSCs.

In some embodiments, it is confirmed that the spheroids of the iPSCs formed according to the method of the present inventive concept each have a greater diameter and a more spherical-like shape than spheroids formed according to other methods. In addition, it is confirmed that the spheroids of the iPSCs formed according to the method of the present inventive concept show an increase in the number of spheroids having a diameter of about 200 μm or more, compared to spheroids formed according to other methods.

In the spheroids of the iPSCs formed according to the method of the present inventive concept, the iPSCs exhibit enhanced pluripotency. In some embodiments, in the spheroids of the iPSCs formed according to the method of the present inventive concept, it is confirmed that the expression level of the pluripotent expression marker (for example, Oct-4 and Sox-2) is higher than that of the pluripotent expression marker in the iPSCs cultured in a petri dish or a flat Si substrate induced pluripotent stem cell.

In addition, when the iPSCs derived from the spheroids formed in an embodiment are subjected to differentiation, it is confirmed that all the markers of the 3 germ layers are expressed. That is, it is confirmed that the iPSCs in the spheroids formed according to the method of the present inventive concept maintain differentiation potency.

Hereinafter, the present inventive concept will be described in further detail with reference to the following examples. However, these examples are for illustrative purposes only and are not intended to limit the scope of the one or more embodiments.

Methods

1. Production of iPSCs

Secondary mouse embryonic fibroblasts (MEFs) containing a doxycycline (DOX)-inducing OSKM factor (for example, Oct3/4, Sox2, and Klf4 alc c-Myc) were prepared. In terms of a reprogramming process, the secondary MEFs (from the third and fourth subcultures) were plated at density of 1.5-2×10⁴ cells/cm² onto a Geltrex-coated culture dish containing a MEF-conditioned medium (i.e., a Dulbecco's Modified Eagle's medium (DMEM) supplemented with 10% FBS, 0.1 mM NEAA, and 2 mM Glutamax). In the following day (0^{th} day), DOX (Sigma-Aldrich; 2-8 μg/mL) was treated thereon for the first time, and then, the same treatment was continued until the 13th day of the culture. The resulting secondary MEFs were cultured for a few more days in an additional MEF-conditioned medium, followed by being modified in an initial medium (RepM-Ini; a knock-out DMEM supplemented with 10% knock-out serum replacer, 5% FBS, 0.1 mM NEAA, 2 mM Glutamax, and 0.055 mM βmercaptoethanol) for 3 days to be subjected to a reprogramming process. In terms of cell proliferation, Nanog-GFP positive colonies were collected. These colonies were maintained in a RepM-Ini medium containing 1,000 units/ml of leukemia inhibitory factor (LIF) and 2i (ERK1/2 and glycogen synthase kinase 3 inhibitor).

2. Preparation of Nanocolumn Array

2.1. Preparation of Nanocolumn Array

A Si substrate (i.e., a Si-wafer) (100, p-type, boron-doped, 1-30 Ωcm-1, Taewon Scientific, South Korea) was ultrasonically washed by using acetone and isopropyl alcohol for 5 minutes, followed by being dried by nitrogen injection. An oxygen plasma treatment (Femto Science, South Korea) was performed on the Si substrate for 5 minutes to activate it. Then, the activated Si substrate was immersed in a polycationic solution (prepared by dissolving 0.03 g of poly(arylamine hydrochloride) (PAH) (a molecular weight of 15,000, Aldrich) in 15 ml of a 2.0 M NaCl aqueous solution) for 5 minutes, to thereby deposit polyelectrolyte multi-layers by layer-by-layer (LbL) deposition. Afterwards, the Si substrate was sequentially immersed in deionized water for 1 minute and in a polyanionic solution (prepared by dissolving 0.03 g of poly(sodium 4-styrene sulfonate (PSS) (a molecular weight of 70 000, Aldrich) in 15 ml of a 2.0 M NaCl aqueous solution) for 5 minutes. After the Si substrate was rinsed by using deionized water, the LbL deposition was repeated until the Si substrate was coated with designated polyelectrolyte multi-layers, for example, in the stated order of PAH-PSS-PAH-PSS-PAH. To make a pattern on a gold surface, the Si substrate was coated with diluted colloids of polystyrene (PS) nanospheres. Then, regions not coated with the PS nanospheres were removed by a reactive ion-etching process. A metallic film having a thickness of 20 nm was deposited on the Si substrate decorated with PS nanospheres by silver shot through vacuum heat deposition (1-5 mm, 99.9%, Alfa Aesar). Afterwards, to remove the PS nanospheres, the Si substrate was subjected to sonication in ethanol for 1 minute. At this step, the Si substrate was immersed in a Si-etching solution, and then, was evenly covered with a gold thin film including a random array with nanosized holes where perpendicular nanocolumns are to be formed. After the gold-covered Si substrate was thoroughly washed by using deionized water, the gold-covered Si substrate was instantly immersed in a mixed etching solution containing hydrofluoric acid (4.6 M) and peroxide (0.44 M). Afterwards, the gold-covered Si substrate was thoroughly rinsed three times by using deionized water, followed by being dried with blowing nitrogen. Finally, the resultant substrate was immersed in boiling aqua regia for 10 minutes, so as to remove remaining Ag flakes, and then, was rinsed three times by using deionized water, followed by being dried with blowing nitrogen.

2.2. Formation of Nanocolumn Array (vSNA) Pattern

To prepare a negatively-patterned vSNA substrate, a photoresist pattern was immobilized on the pre-washed Si substrate, prior to polyelectrolyte deposition and a subsequent Si-etching process. Briefly, a Si substrate was treated with hexamethyl disilazane (HMDS), and then, coated with a positive photoresist layer (GXR601, Microchem, USA) at a speed of 4,000 rpm for 35 seconds, followed by being subjected to a mild heat treatment at a temperature of 95° C. for 60 seconds. After being exposed to UV through a pre-determined photomask, the substrate was immersed in a developer solution (MIF 300, Microchem, USA) for 30 seconds. Then, the substrate was rinsed by using deionized water, and dried with blowing nitrogen. To prepare a positively-patterned vSNA substrate, a photoresist pattern was immobilized on a non-patterned vSNA substrate, in a similar manner as described above. Afterwards, the photoresist-immobilized vSNA substrate was immersed in a 0.1 M KOH solution, thereby removing non-buried vSNA.

2.3. Surface Modification of Nanocolumn Array (vSNA)

In terms of appropriate cell attachment, (3-amonopropyl)trimethoxysilane (APTMS) (Sigma-Aldrich) was used to functionalize the vSNA and the Si wafer so that the surface of the vSNA and Si wafer was covered with a positively-charged amine group. An oxygen plasma treatment was performed on the vSNA and the Si wafer for 10 minutes, thereby exposing hydroxyl groups on the surface of the vSNA and Si wafer. Acetic acid was used as a catalyst, and silanization was performed on the vSNA and Si wafer at room temperature using APTMS in methanol for 30 minutes. The substrates that underwent the treatment was washed with methanol and water, and then, dried with nitrogen gas. To avoid any contamination, the substrate was rinsed three times with Dulbecco's phosphate buffered saline (DPBS), prior to cell seeding.

3. Cell Staining Using CFDA

About 8×10³ iPSCs were seeded onto a plate coated with an attachment factor (AF), a Si wafer, and a vSNA substrate (each having an identical area of 0.5×0.5 cm²). Afterwards, on day 3 of the culture, the iPSCs were stained using 1 μM CFDA (Sigma-Aldrich). A culture medium including the cell-plated substrate was supplemented with a CFDA solution, and then, was incubated for 10 minutes. The cell-plated substrate was washed with PBS, to be subjected to imaging. Then, fluorescence images of the cells were obtained by using a fluorescent microscope (Axioskop2 FS plus; Carl Zeiss, Oberkochen, Germany). The cells stained with CFDA were visualized based on the fluorescent images collected at wavelengths of 528±19 nm (excitation at wavelengths of 490±10 nm).

4. MTS Assay

Cell viability was assessed using CellTiter 96 Aqueous One Solution Cell Proliferation Assay (MTS assay, Promega) according to the protocol provided by the manufacturer. Briefly, on day 1, cells residing on various substrates were seeded into 48-well plates, each well containing fresh media supplemented with 20% MTS solution, and the plates were incubated at a temperature of 37° C. for 3 hours. The changes in absorbance of the incubated cells were read at 490 nm using a spectrophotometric plate reader.

5. Staining Using CFDA/Propidium Iodide (PI)

About 8×10³ iPSCs were seeded onto each of a petri dish and a vSNA (at a density of 0.5×0.5 cm² per dish and array), and then, incubated for 5 days. Aggregates generated therefrom were plated on an AF-coated dish, and cultured overnight so as to be attached to the dish. A culture medium was supplemented with a 1 μM CFDA solution and 1 μM PI, and then, was incubated for 10 minutes. The cell-plated substrate was washed twice with phosphate buffered saline (PBS), before being subjected to imaging. Then, fluorescence images of the cells were obtained by using a confocal scanning laser microscope (FV1000; Olympus, Hamburg, Germany).

6. Immunoblotting

Cells were collected and lysed in a buffer solution (containing 40 mM Tris-HCl, pH 8.0, 120 mM NaCl, 0.1% Non-idet-P40, 100 mM phenylmethylsulfonyl fluoride, 1 mM sodium orthovanadate, and a protease inhibitor cocktail). Proteins were separated by SDS-polyacrylamide gel electrophoresis, and then, were transferred onto polyvinylidene difluoride (PVDF) membranes. The PVDF membranes were each blocked with 5% nonfat dry milk in Tris-buffered saline containing 0.1% Tween-20, and then, were incubated with primary antibodies overnight at a temperature of 4° C. After peroxidase-conjugated secondary antibodies were used for color development, immunoblots were visualized by enhanced chemiluminescence (ATTO KOREA, Korea).

7. Semiquantitative RT-PCR

For gene expression analysis, total RNA was extracted using an RNeasy Mini Kit (QIAGEN, USA). From the extracted RNA, cDNA was synthesized using a TOPscript™ cDNA Synthesis kit (Enzynomics, Korea). The primers provided with the Mouse/Rat Pluripotent Stem Cell Assessment Primer Pair Panel (R&D System, UK) were used. The detailed procedure for PCR included 30-35 cycles of denaturation at 94° C. for 45 s, annealing at 55° C. for 45 s, and elongation at 72° C. for 45 s. The primers for mouse Rex-1, synthesized by Macrogen (Korea), were 5′-AAGCAGGATCGCCTCACTGT-3′ (forward) and 5′-GCTTCCAGAACCTGGCGAGA-3′ (reverse). The detailed procedure for PCR included 25 cycles of denaturation at 94° C. for 45 s, annealing at 58° C. for 45 s, and elongation at 72° C. for 45 s.

8. FACS Analysis of SSEA-1

Accutase (Life Science) was used to break up spherical colonies of the iPSCs to single cells of smaller sizes. After being rinsed with PBS, the cells were fixed with 4% paraformaldehyde, permeabilized with 0.2% Triton X-100 for 10 minutes, and exposed to 2% bovine serum albumin (BSA) for 30 minutes. The cells were incubated with mouse polyclonal antibodies against SSEA-A (diluted at 1:100; Santa Cruz Biotechnology, Santa Cruz, Calif.) for 1 hour at room temperature, washed three times with PBS, and incubated with tetramethylrhodamine isothiocyanate (TRITC)-conjugated goat antibodies against mouse immunoglobulin M (diluted at 1:200; Molecular Probes) for 30 minutes at room temperature. Afterwards, to measure the average density of the fluorescence, cells (20,000 cells per sample) were subjected to FACS analysis using a Guava EasyCyte™ flow cytometer (Millipore).

9. Differentiation Analysis

In terms of differentiation analysis, the spheroids of the iPSCs that were cultured for 5 days either in the nanocolumn array (vSNA) or petri dish, and then transferred onto a AF-coated dish. Then, the cells were cultured in an EB medium supplemented with 10% FBS (a knock-out DMEM supplemented with 15% knock-out serum replacer, 0.1 mM NEAA, and 2 mM Glutamax) for 2 weeks. Here, the medium was replaced with spent medium every two days. The total RNA was purified, and the gene expression was analyzed by the semiquantitative RT-PCR described above.

10. Immunofluorescence Staining with F-Actin, and Vinculin

After washing the sample with PBS once, the sample was fixed with 4% formaldehyde solution in PBS for 20 minutes. Then, a washing process using PBS was performed thereon three times. The cells were permeabilized with 0.2% Triton X-100 for 10 minutes, and then, were washed three times with PBS. The sample was blocked with 2% BSA in PBS for 30 minutes at room temperature, and then, was incubated with anti-vinculine primary antibodies (Sigma-Aldrich; 1:100) for 60 minutes. Afterwards, the sample was incubated with Alexa-488-conjugated secondary antibodies (Invitrogen; 1:200) for 1 hour at room temperature. TRITC-conjugated phalloidin (Sigma-Aldrich; 25 mg/ml) was added thereto in terms of visualizing F-actin. Each step was performed in PBS containing 2% BSA, and the cells between the steps were washed with the same solution three times for 5 minutes. Then, the integrity of the cell nucleus was confirmed by Hoechst (Invitrogen) staining. Here, all images were obtained by using a confocal scanning laser microscope (FV1000; Olympus, Hamburg, Germany).

11. Cell Attachment Assay

About 8×10³ iPSCs were seeded into each of a AF-coated dish, a Si wafer, and vSNA (at a density of 0.5×0.5 cm² in all cases), and then, incubated for 24 hours. A culture medium including the cell-plated substrate was supplemented with a CFDA solution, and then, was incubated for 10 minutes. The cell-plated substrate was washed twice with PBS. Then, fluorescence images of the cells were obtained by using a fluorescent microscope (Axioskop2 FS plus; Carl Zeiss, Oberkochen, Germany). The cells stained with CFDA were visualized based on the fluorescent images collected at wavelengths of 528±19 nm (excitation at wavelengths of 490±10 nm).

Results

1. Inquest on Characteristics of Spheroids of iPSC Formed on Nanocolumn Array

To investigate influences of the nanocolumn array on the characteristics of the mouse iPSCs that were cultured in the vSNA, 6 different vSNAs were prepared. Physical characteristics of the prepared vSNAs are shown in Table 1. Then, the mouse iPSCs were seeded onto each of the prepared vSNAs, an AF-coated plate, a petri dish, and a Si wafer, and then, were cultured therein.

TABLE 1
Physical characteristics of vSNAs

| | Diameter (bottom | Density | Length |
|---|---|---|---|
| | surface, nm) | (per 100 μm²) | (μm) |
| Short | 170 ± 4 | 155 ± 5 | 1.0 ± 0.010 |
| Medium | 160 ± 4 | 163 ± 24 | 1.8 ± 0.080 |
| Long | 161 ± 7 | 110 ± 4 | 4.0 ± 0.36 |
| vSNA1 | 170 ± 3 | 29 ± 2 | 0.60 ± 0.010 |
| vSNA2 | 165 ± 4 | 155 ± 5 | 1.0 ± 0.010 |
| vSNA3 | 117 ± 13 | 396 ± 35 | 0.70 ± 0.030 |
| vSNA4 | 136 ± 8 | 710 ± 2 | 0.80 ± 0.040 |

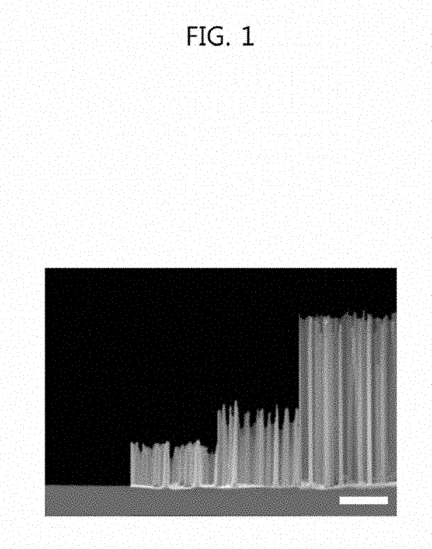

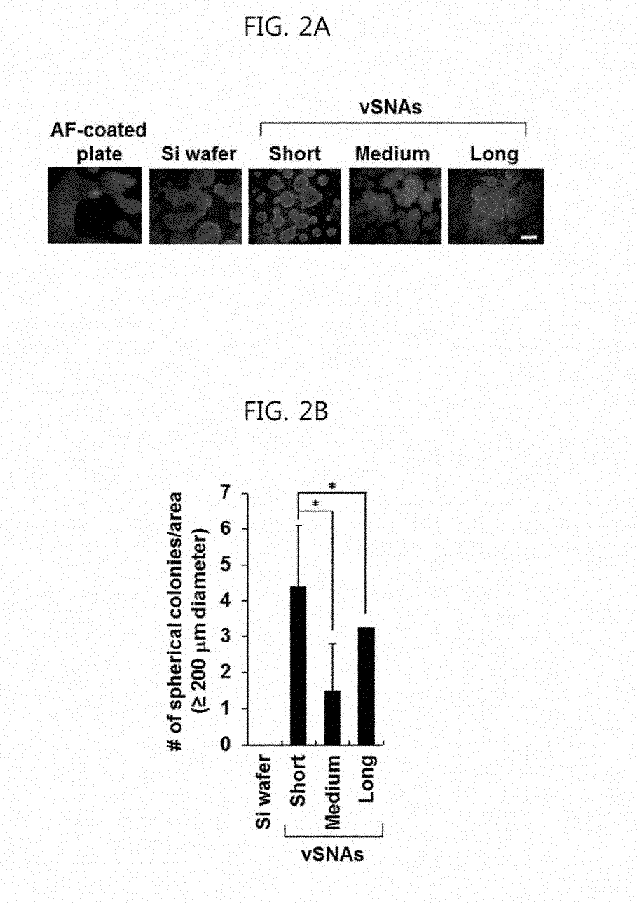

First, it was examined whether the length of the nanocolumn on the vSNAs influenced the shape of the iPSCs. Based on the scanning electron microscope (SEM) images, the average length of the vSNAs was measured, and consequently, the length of the nanocolumn on the ‘Short vSNA’ was measured to be about 1.0 μm, the length of the nanocolumn on the ‘Medium vSNA’ was measured to be about 1.8 μm, and the length of the nanocolumn on the ‘Long vSNA’ was measured to be about 4.0 μm (see FIG. 1). The average diameter of these three vSNAs were about 160 nm or less, and the three vSNAs had a similar density of about 150/100 μm² (the number of nanocolumns/array area). The cell-surface area of the iPSCs cultured in each of the vSNAs for 3 days was observed as being stained with a viability marker, CFDA. Consequently, as shown in FIG. 2A, the iPSCs cultured on the Si wafer had flat colonies formed therein in a similar manner as in the AF-coated plate, whereas the iPSCs cultured on the vSNA all showed spherical shapes and numbers of colonies formed therein. In addition, the spherical colonies having a diameter of about 200 μm or more were more likely to be found in the Short vSNA than in the Long vSNA and the Medium vSNA (see FIG. 2B). The results imply that the nanocolumn of short length formed vertically on the substrate provides favorable environment for formation of spherical colonies.

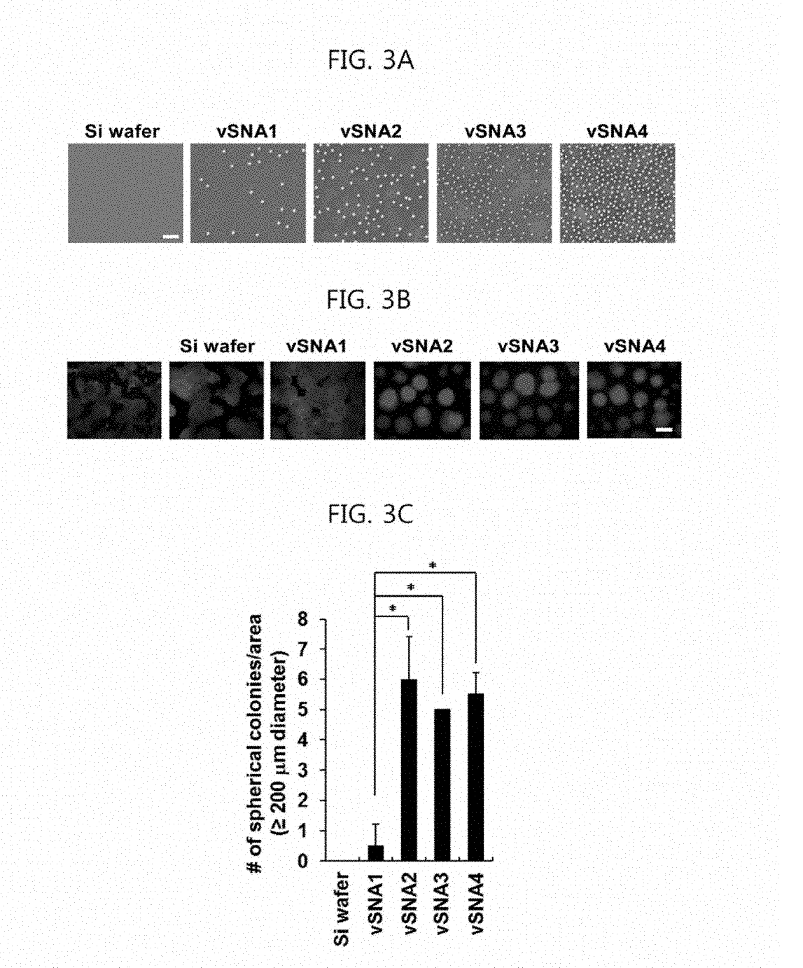

In addition, the effects of the vSNA density on the formation of the spherical colonies of the iPSCs were examined. Here, the average density of the vSNAs was measured by the SEM images (see FIG. 3A). The vSNAs were found to have a similar diameter and a similar length (1.0 μm). It is also confirmed that the spherical colonies of the iPSCs having a large diameter 200 μm) were formed in great numbers in vSNAs having high density (>100 vSNs/100 μm²; vSNA2, vSNA3, and vSNA4), compared to vSNA having low density (<100 vSNs/100 μm²; vSNA1) (see FIG. 3B and FIG. 3C). In the vSNAs having high density (e.g., vSNA2, vSNA3, and vSNA4), there was so significant differences in the size and number of spherical colonies of the iPSCs. Upon such results above, the vSNA may directly influence the shape of the colonies, and it was also suggested that the short length and high density of the vSNA provide favorable environment for the formation of spherical colonies of the iPSCs.

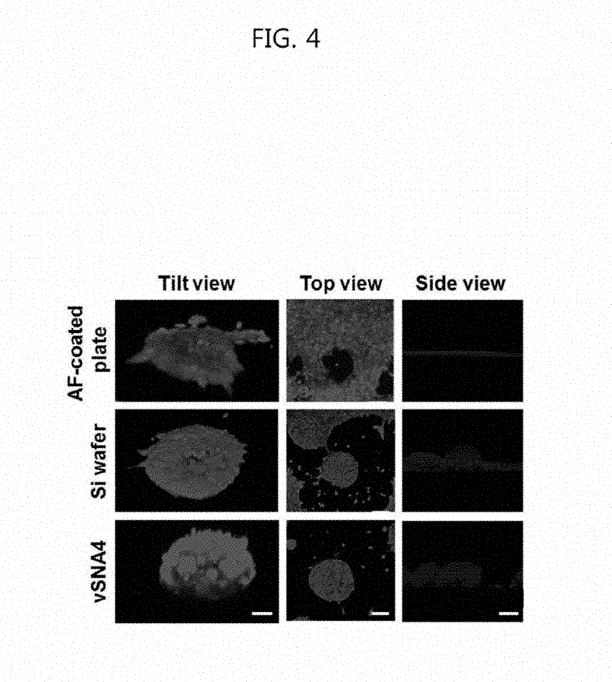

A 3D image of the spherical colonies of the iPSCs cultured in high-density vSNA was constructed by z-sectioned confocal fluorescence images. For this purpose, vSNA having a diameter of about 136 nm or less, a length of about 0.8 μm or less, and a density of about 710 vSNs/100 μm² or less (vSNA4) was used. The iPSCs cultured in the vSNA4 were found to increase the number and size of the colonies, and also produced a 3D structure in a spherical form. In addition, the growth of the colonies continued until the colonies were very tightly packaged. Meanwhile, cells cultured on the AF-coated plate and the Si wafer were found to be attached to the surface of the substrates and form flat colonies (see FIG. 4). Based on such results above, it was confirmed that, compared to the culture container having a flat substrate there was increased formation of spheroids of the iPSCs on VSNAs. In addition, it was confirmed that the nanocolumn array having high density of about 150/100 μm² or more and the short length of about 1.5 μm or less provides favorable environments for inducing production of spheroids having a larger size and formed in a more spherical-like form.

2. Morphological Analysis on Colonies of iPSCs Cultured on Nanocolumn Array

First, the MTS assay was used to evaluate viability of the iPSCs cultured on vSNA following 24-hour incubation. MTS refers to a tetrazolium compound that is to be reduced to formazan, which is a blue metabolite in mitochondria. That is, the amount of formazan produced by the cells is proportional to the viable cells. The viability of the cells was similarly shown in each of the culture substrate (i.e., AF-coated plate, petri dish, Si wafer, and vSNA4, data not shown). The results obtained therefrom include data of vSNA causing no change in the cell viability and having no toxicity.

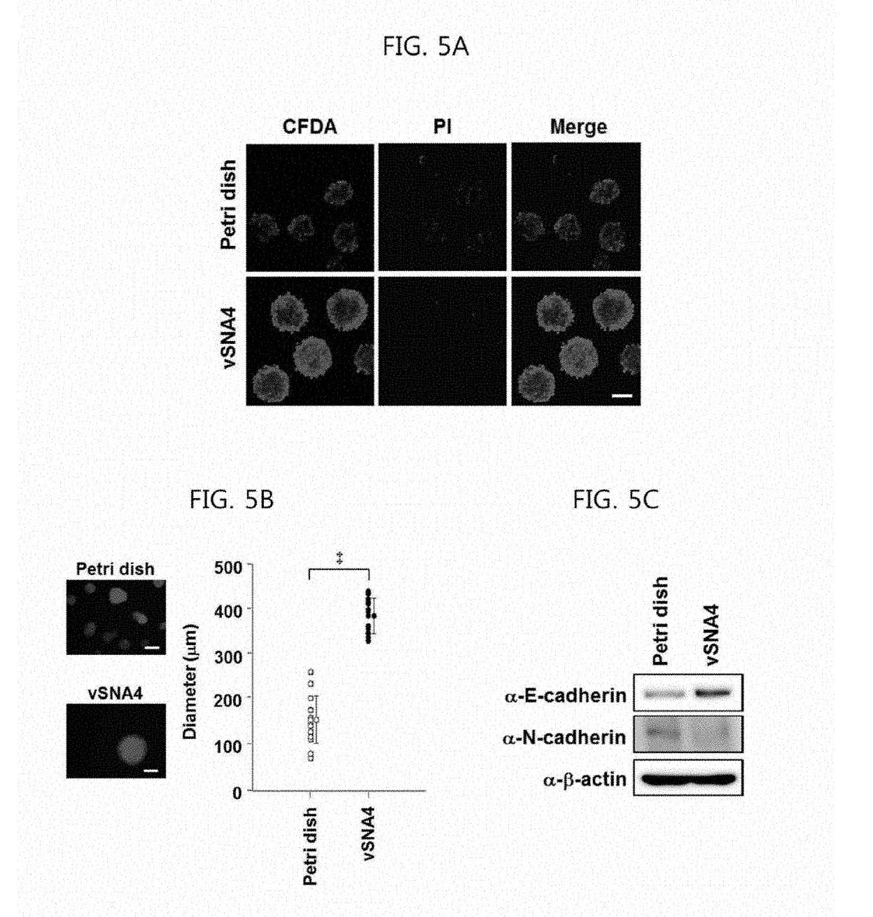

In addition, the ratio of live and dead cells in the spherically-formed colonies cultured for 5 days on the vSNA was examined by employing CFDA/PI staining and compared with the conventional methods (suspension culturing on a petri dish). The viable cells showed an extensive CFDA labelling, whereas the dead cells were not stained with CFDA and visualized by PI. Here, interestingly, most cells in the spherical colonies incubated for 5 days on vSNA4 or petri dishes were viable as stained by CFDA, whereas the cell death indicated by PI staining was more pronounced in the petri dishes than on vSNA4 (see FIG. 5A).

Furthermore, the diameter of the spherical colonies cultured on the vSNA for 5 days was measured. As shown in FIG. 5B, the iPSCs cultured on the vSNA had a greater diameter than that of the cells cultured on the petri dish, and the spherical colonies cultured on the vSNAp produced more compact spheroids that those of the cells cultured on the petri dish. Such results confirm that the vSNA reduces the cell death during the culturing of the spheroids. In addition, E-cadherin, which is a cell-to-cell adhesion protein, was expressed at a higher level in the spheroids of the iPSCs cultured on the vSNA than in the spheroids of the iPSCs cultured on the petri dish (see FIG. 5C), whereas N-cadherin was expressed at a lower level in the spheroids of the iPSCs cultured on vSNA than in the spheroids of the iPSCs cultured on the petri dish (see FIG. 5C). That is, it was confirmed that the vSNA leads to enhanced cell-to-cell interactions upon induction of the formation of compact spheroids. Based on the results above, it was confirmed that increased expression of E-cadherin may be related to maintenance of the pluripotency of the iPSCs on the vSNA.

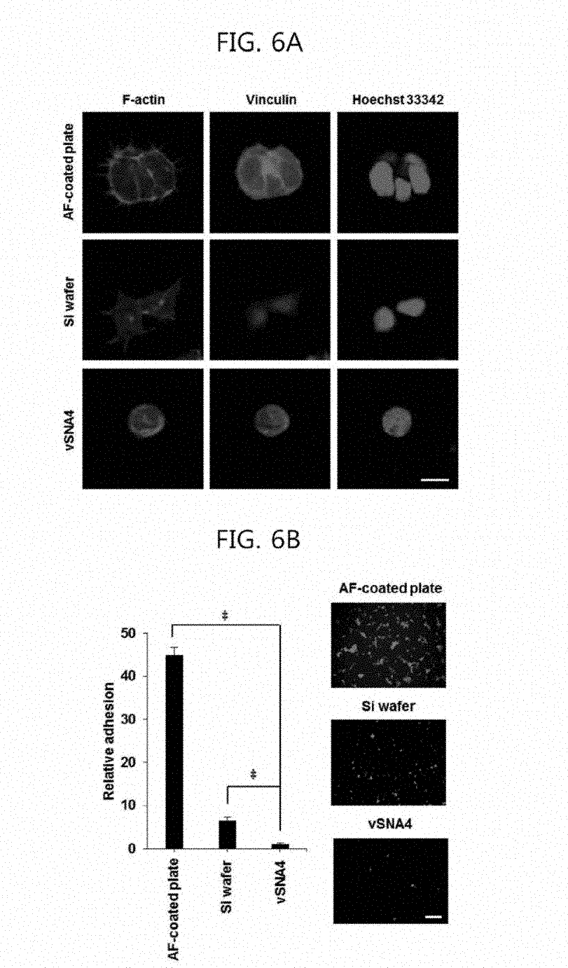

To evaluate surface-to-cell interactions of the iPSCs, the distribution of cytoskeletal actin and focal adhesion vinculin, which is responsible for the maintenance of the cell shape and the movement of the cells on the attached surface, were examined. FIG. 6A shows immunofluorescent image of filamentous actin (F-actin) and vinculin on various substrates. Interestingly, the iPSCs cultured on vSNA4 appeared much smaller and more rounded than did those on AF-coated plates. More remarkably, the fluorescent F-actin staining manifested many cellular protrusions on both the AF-coated plate and the flat Si, whereas such localized F-actin structures were hardly found on vSNA4. Relative cellular adhesion was also assessed by adhesion retention after several washing steps using a PBS solution. While most iPSCs on vSNA4 were readily rinsed away, those on AF-coated plates were mostly retained (FIG. 6B). The results above imply that the vSNA responsible for weakening the cell adhesion induces spherical cell aggregates in a spontaneous manner.

3. Analysis on Expression of Pluripotency Marker of the iPSCs Cultured on vSNA

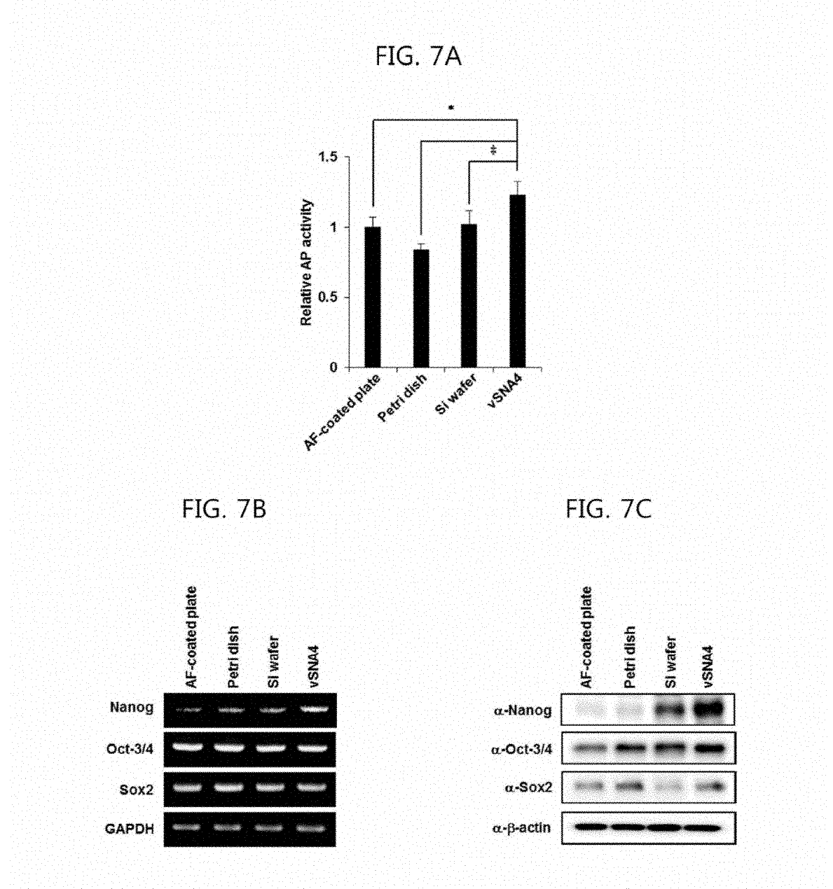

It was examined whether the formation of the spherical colonies on the vSNA influenced the pluripotency of the iPSCs. Alkaline phosphatase (AP) used herein is a hydrolyze enzyme responsible for removing phosphate groups from nucleotides, proteins, and alkaloids. AP is known to be expressed at high levels in embryonic stem cells (ESCs) and other pluripotent stem cell types, such as iPSCs. Thus, after culturing cells on various culture substrates (i.e., AF-coated plate, petri dish, Si wafer, and vSNA) for 5 days, the activity of AP in the iPSCs was analyzed. As shown in FIG. 7A, the activity of AP was elevated in the iPSCs cultured on the vSNA compared to that in the iPSCs cultured on other culture surfaces.

In addition, mRNA expression levels of pluripotency-related transcription factors, such as Nanog, Oct-3/4, and Sox-2, were analyzed by semi-quantitative RT-PCR. By comparing with the iPSCs cultured on the AF-coated plate, the petri dish, or the Si wafer, the iPSCs cultured on the vSNA showed significant up-regulation of Nanog (see FIG. 7B). To figure out whether the expression level of Nanog was high in the iPSCs cultured on the vSNA, compared to other substrates, immunoblot assay was performed (see FIG. 7C).

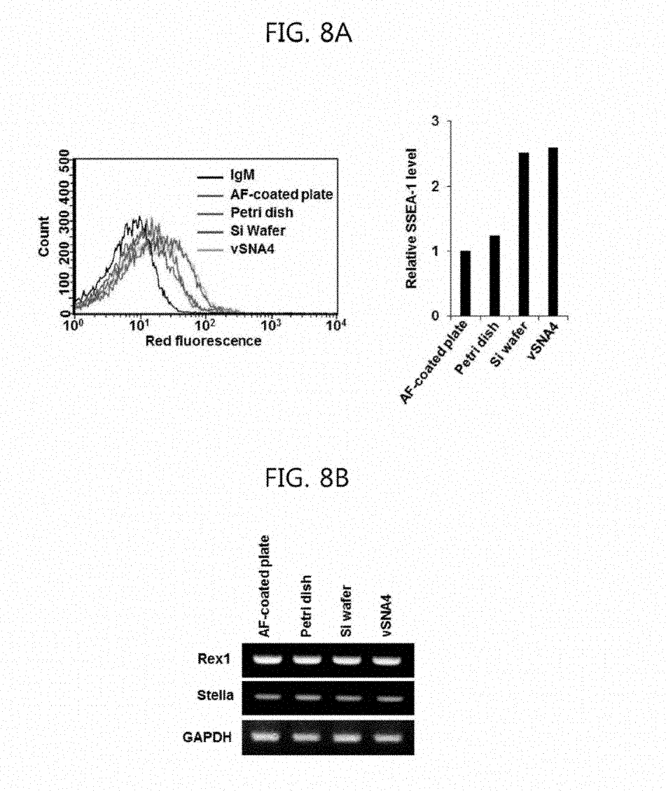

Additionally, flow cytometry-based quantitative analysis confirmed that the expression of stage-specific embryonic antigen (SSEA)-1, a typical pluripotency marker of mouse iPSCs, was most pronounced in the iPSCs harvested from vSNA4 (FIG. 8A). The expression levels of naïve pluripotency genes such as Stella and Rex1 did not substantially vary among the iPSCs grown on all four substrates, suggesting that the undifferentiated state was maintained in the iPSC colonies on vSNA4 (FIG. 8B).

4. Analysis of Differentiation Potency of iPSC Cultured on vSNA

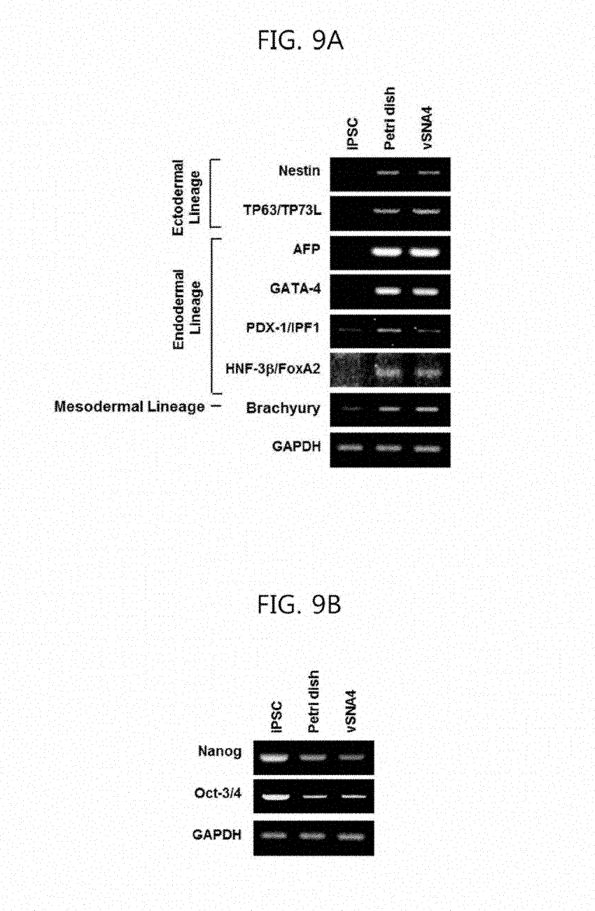

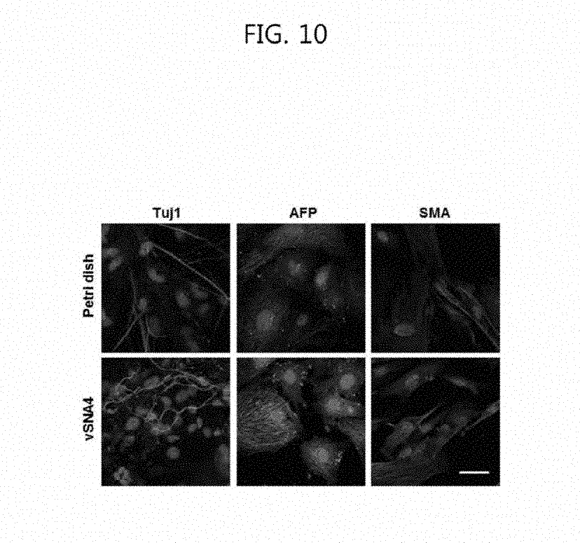

The iPSCs cultured on the vSNA4 were subjected to in vitro differentiation assay to examine potential differentiation capability. Based on the analysis on the expression of the transcripts, the induction of the expression of the initial lineage marker with respect to the 3 germ layers was confirmed (see FIG. 9A; labeled endoderm, labeled ectoderm, and labeled mesoderm). Consequently, it was confirmed that the iPSCs cultured on the vSNA maintained potential differentiation capability and the expression of pluripotent markers were decreased as the iPSCs were subjected to differentiation (FIG. 9B). In addition, the three germ layers were identified based on protein immunofluorescent images of differentiation markers, Tuj1 (etcoderm), AFP (endoderm), and SMA (medoderm). FIG. 10 shows immuno-fluorescent images of three germ layers of the iPSCs by each differentiation marker stained differently, wherein the iPSCs were cultured on the petri dish and vSNA4 for 14 days.

The results above imply that the iPSCs cultured on the vSNA have ability to be differentiated into cells representing the three germ layers. In addition, these data imply that the culture system on the vSNA supports the pluripotency of the iPSC.

5. Confirmation of Possibility of Recycling vSNA and Ability of Culturing Large-Scale Cells

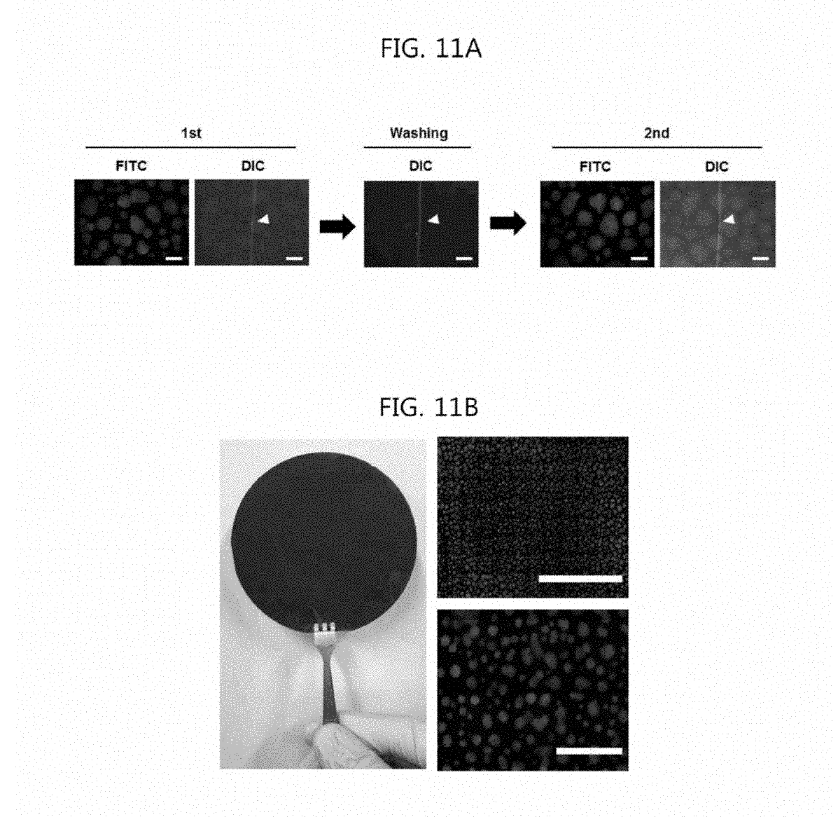

FIG. 11A shows fluorescent microscope images and DIC images of the spheroids of the iPSCs, the spheroids being formed by culturing the iPSCs as new cells in the same nanocolumn as the one used for culturing and washing iPSCs. Here, the iPSCs were washed out to remove the previously formed colonies therefrom. It was confirmed that the spheroids of the newly cultured iPSCs were repeatedly formed on the vSNA used for the culturing while maintaining morphological characteristics of the spheroids.

FIG. 11B shows a picture of the scale-upgraded nanocolumn array to be placed on a culture dish having a diameter of 75 mm and fluorescent images of the colonies of the iPSCs cultured on the nanocolumn array. Here, it was confirmed that the colonies of the iPSCs were well formed in the spherical form throughout the nanocolumn array.

Accordingly, it was confirmed that the nanocolumn array according to the present inventive concept was utilized for large-scale culturing of the iPSCs, and furthermore was recyclable.

As described above, iPSCs cultured according to one or more embodiments may have improved stemness, and diameters and the number of spheroids of the iPSCs may be increased. In addition, the iPSCs may have reduced cell death in the spheroids thereof, thereby increasing cell viability. Thus, the present inventive concept may be utilized for culturing the iPSCs. In addition, the culture container may be recyclable and can be used for large-area cell culturing, and thus may be suitable as a culture container for culturing the iPSCs that require a large amount for use in cell therapy applications.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the inventive concept as defined by the following claims.

What is claimed is:

1. A container for culturing an induced pluripotent stem cell, the container having a nanostructure array.

2. The container of claim 1, wherein the nanostructure array has a plurality of nanocolumns that are spaced at random intervals on a substrate.

3. The container of claim 2, wherein the length of each nanocolumn is in a range of about 0.5 μm to about 1.5 μm.

4. The container of claim 2, wherein the density of nanocolumns in the nanostructure array is in range of about 150/100 μm² to about 1,000/100 μm² (the number of nanocolumns/area of nanostructure array).

5. The container of claim 1, wherein the container is designed to culture the induced pluripotent stem cell in an undifferentiated condition under a feeder layer-free.

6. The container of claim 1, wherein the container is for forming a spheroid of the induced pluripotent stem cell.

7. The container of claim 1, wherein the nanostructure array is reusable.

8. A method of culturing an induced pluripotent stem cell under an undifferentiated condition, the method comprising:
culturing an induced pluripotent stem cell in a nanostructure array.

9. The method of claim 8, wherein the nanostructure array has a plurality of nanocolumns that are spaced at random intervals on a substrate.

10. The method of claim 8, wherein the length of each nanocolumn in the nanostructure array is in a range of about 0.5 μm to about 1.5 μm.

11. The method of claim 8, wherein the density of nanocolumns in the nanostructure array is in range of about 150/100 μm² to about 1,000/100 μm² (the number of nanocolumns/area of nanostructure array).

12. A method of forming a spheroid of an induced pluripotent stem cell, the method comprising:
culturing an induced pluripotent stem cell on a nanostructure array.

13. The method of claim 12, wherein the nanostructure array has a plurality of nanocolumns that are spaced at random intervals on a substrate.

14. The method of claim 12, wherein the length of each nanocolumn in the nanostructure array is in a range of about 0.5 μm to about 1.5 μm.

15. The method of claim 12, the density of nanocolumns in the nanostructure array is in range of about 150/100 μm² to about 1,000/100 μm² (the number of nanocolumns/area of nanostructure array).
